(19) United States
(12) Patent Application Publication
(10) Pub. No.: US 2022/0118280 A1
(43) Pub. Date: Apr. 21, 2022

(54) DETERMINING METAMERIC SETTINGS FOR A NON-LINEAR LIGHT SOURCE

(71) Applicant: Oxford University Innovation Limited, Oxford (GB)

(72) Inventor: Manuel SPITSCHAN, Oxford (GB)

(21) Appl. No.: 17/428,073

(22) PCT Filed: Feb. 7, 2020
(86) PCT No.: PCT/GB2020/050278

§ 371 Date: Aug. 3, 2021

(30) Foreign Application Priority Data
Feb. 7, 2019 (GB) 1901723.5

(51) Int. Cl. A61N 5/06; G01J 3/46; H05B 45/22

(52) U.S. Cl. CPC A61N 5/0622; G01J 3/462; G01J 3/465; G01J 2003/2869; A61N 2005/0651; A61N 2005/0663; H05B 45/22

(57) ABSTRACT

There is provided an apparatus and method for determining metameric settings for a nonlinear light source comprising a set of N primaries. Spectral data indicative of a spectral output of each of the N primaries at each of a plurality of predetermined intensity values is received. The method comprises determining, in dependence on the received data, a relationship for each of the N primaries between intensity and spectral output over at least a portion of a visible spectrum; determining a first activation corresponding to activation of a first photoreceptor type and a second activation corresponding to activation of a second photoreceptor type in dependence on the determined relationship for each of the N primaries; and selecting a background set of intensity values and a modulation set of intensity values of the N primaries. The background set and modulation set are selected such that a difference between the determined first activation corresponding to the background set and the determined first activation corresponding to the modulation set is according to a first criteria; and a difference between the determined second activation corresponding to the background set and the determined second activation corresponding to the modulation set is according to a second criteria.

### BACKGROUND

The human retina comprises several different types of photoreceptor exhibiting different spectral sensitivities to light. The spectral output of a light source impacts whether, and to what extent, each of the photoreceptor types is activated. In some circumstances it is desirable to selectively activate or abstain from activating certain photoreceptor types, while maintaining a consistent activation for others. For example, it may be desired to achieve a constant visual response in rod and cone activation, but differing melanopsin activation in order to modulate melatonin production, circadian phase shifting, alertness and other physiological and behavioural functions. In another example it may be desired to achieve a constant melanopsin activation, but differing rod or cone activation to achieve a modulation in perceived brightness or colour, or both.

It is possible to attain a modulation of activation of one photoreceptor type and maintained activation of another experimentally by the construction of metamers. Metamers are two or more stimuli, i.e. spectral outputs from light sources, with the property that they activate a first set of photoreceptors in the same way but differ in how much they activate a second set of photoreceptors. Metamers comprise a number of primaries, which are defined as spectrally independent channels within the light source whose output may be independently controlled or tuned. Each primary, given an input intensity I, will produce a given spectral output S(λ,I), which in turn will achieve a corresponding activation P for each photoreceptor type.

Metamers for a light source comprising N primaries may be constructed by determining a first set of input intensity values (I_{1,1} . . . I_{N,1}) and a second set of input intensity values (I_{1,2} . . . I_{N,2}) for each of the N primaries which attain a desired difference in activation P₁ for the first set of photoreceptors and a desired difference in activation P₂ for the second set of photoreceptors. It is therefore important to be able to accurately characterise the activation P of each photoreceptor given any set of input intensity values (I₁ . . . I_{N}) for each of the primaries.

It may be desired to create metamers using non-linear light sources, such as modern LEDs as primaries, which exhibit spectral shifts as a function of input intensity. Mapping from the input intensity values of said non-linear light sources to the activation of each photoreceptor is non-trivial, causing inaccuracy in the construction of metamers.

It is an object of embodiments of the invention to at least mitigate one or more of the problems of the prior art.

### BRIEF DESCRIPTION OF THE DRAWINGS

Embodiments of the invention will now be described by way of example only, with reference to the accompanying figures, in which:

FIG. 1 shows a schematic illustration of a system according to the present invention;

FIG. 2 shows a gamma curve relating input intensity to output of an example primary of a light source;

FIG. 3 illustrates a difference between a linear and a non-linear primary for a light source;

FIG. 4 shows a method 400 according to the present invention for determining a relationship between input intensity and spectral output for a light source;

FIG. 5 shows a method 500 according to the present invention for determining metameric settings for a light source;

FIG. 6 shows an illustration of example spectral sensitivity of a plurality of photoreceptor types;

FIG. 7 shows a method 700 according to the present invention for determining metameric settings for a light source;

FIG. 8 shows different manifestations of a spectral shift effect for different non-linear primaries; and

FIG. 9 illustrates an example inferred spectral output for a primary.

### DETAILED DESCRIPTION OF EMBODIMENTS OF THE INVENTION

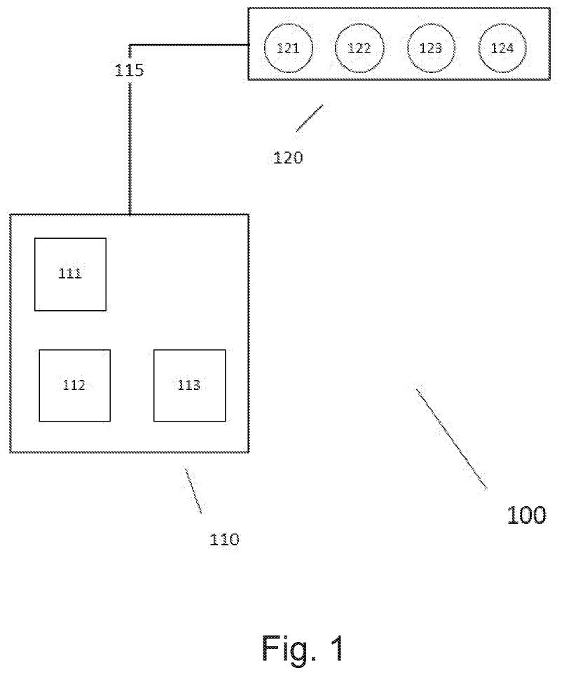

FIG. 1 illustrates a system 100 according to an embodiment of the invention. The system 100 comprises a light source 120 and a computer 110 arranged to determine one or more settings for the light source 120.

The computer 110 comprises one or more memory devices 111 for data storage and one or more processors 112 configured to operably execute computer readable instructions to operably implement a method according to an embodiment of the invention on the computer 110. The computer readable instructions may be stored for example on the one or more memory devices 111. The computer 110 may further comprise one or more interfaces 113 comprising an input device for receiving user input and/or an output device for outputting information to a user, for example via a display or speaker. The computer 110 may be communicably coupled to one or more networks such as the internet and be operable to receive and transmit data over the one or more networks.

The computer 110 may be communicable to the light source 120, for example over the one or more networks or through a wired input/output (I/O) interface 115. The one or more processors 112 are configured to, in use, determine one or more settings for the light source 120. The computer 110 may then be configured to transmit the one or more settings to the light source 120 in order to configure the light source 120 according to the received settings. Alternatively or additionally, the computer 110 may be configured to store the one or more settings in the one or more memory devices 111.

The light source 120 comprises a set of N primaries 121, 122, 123, 124, . . . , 12N. FIG. 1 illustrates the light source 120 comprising four primaries 121, 122, 123, 124, however the system 100 is not limited to this number. For example, the light source 120 in some embodiments may comprise three primaries 121, 122, 123. Each of the N primaries is a different spectral channel in the light source 120 having a distinct spectral output. The power or input intensity supplied to each primary may be independently configured. Each primary may be an individual output device, or a channel within a single device. For example, each primary 121, 122, 123 may be an individual LED or a channel within an LED. For example, RGB systems employ three primaries corresponding to Red, Green and Blue channels, although other systems can be envisaged to produce different spectral outputs. For example, other light sources may comprise different numbers of primaries or primaries having different output wavelengths.

In some embodiments, use of more than three primaries may be required. Given N photoreceptor classes, it is possible to stimulate one photoreceptor class and keep the activation of N−1 photoreceptor classes constant by using a light source comprising at least N independent primaries. This requirement relates to the determination of metamers, as will be explained. It is often desired to stimulate melanopsin and keep the activation of the L, M and S cone cells constant. In these embodiments, given the four photoreceptor classes involved, the light source 120 comprises at least four primaries 121, 122, 123, 124.

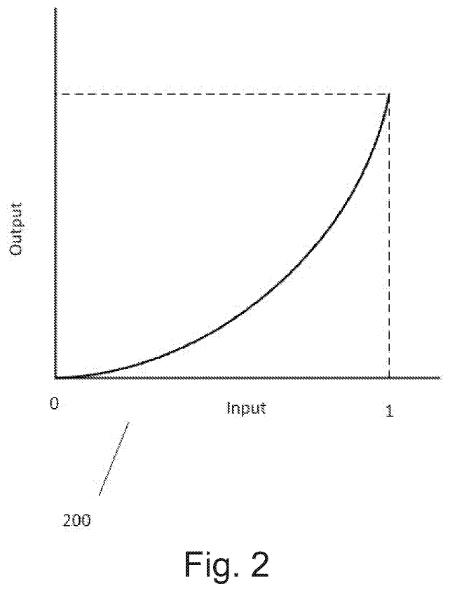

For each primary 121, 122, 123, 124 an input intensity corresponding to an input setting for the primary can be related to a corresponding output radiance emitted from the primary. The input intensity may for example be controlled by controlling a drive current for the respective primary, or by controlling an input signal in some other way, for example by pulse-width modulation (PWM) or pulse-frequency modulation (PFM). Other methods may be used to control the input intensity of the primary, for example via optical filtering or reflectance control. The relationship between input intensity and output radiance of the primary can be illustrated on what is referred to as a gamma curve, as illustrated in FIG. 2.

FIG. 2 shows an example gamma curve 200 indicative of a relationship between input intensity and output radiance for an example primary. The input intensity may be characterised as a fractional number between 0 and 1, 1 being a maximum input intensity. Increasing the input intensity does not necessarily result in a corresponding linear increase in output radiance. The relationship is usually non-linear, as is the case for example gamma curve 200. Each primary will also be characterised by an output spectrum, i.e. its output as a function of wavelength.

If the gamma curve of each primary 121, 122, 123, 124 is known, and its output spectrum is invariant, it is possible to predict all possible spectra that the light source 120 can output. Given the input intensity of each primary, the output spectrum of the whole light source 120 is given by a linear combination of the output spectrum of each individual primary scaled by the output radiance according to the gamma curve for the given input intensity.

Embodiments of the present invention are directed particularly to non-linear light sources 120 comprising a plurality of primaries 121, 122, 123, 124 exhibiting a further non-linearity. In addition to the above non-linearities in the gamma curve, primaries of non-linear light sources further exhibit spectral shifts as a function of input intensity.

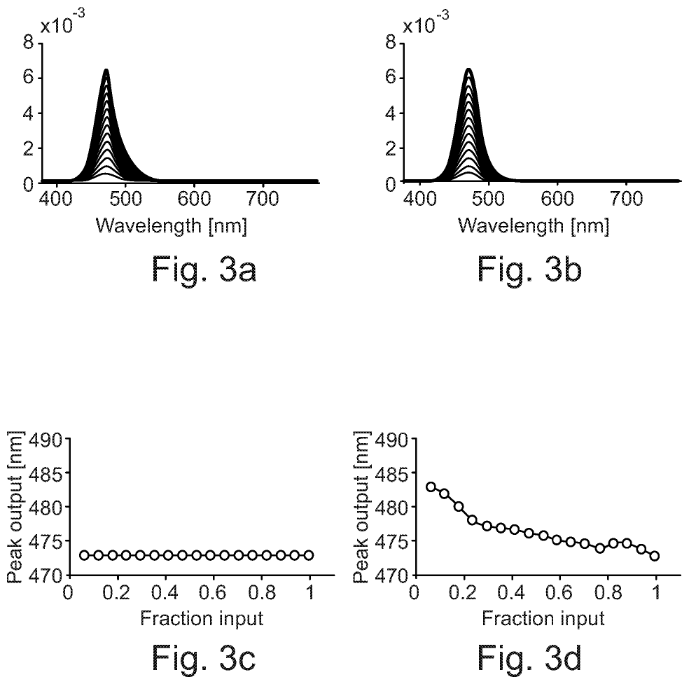

FIG. 3 illustrates such a spectral shift effect. FIG. 3a illustrates an output spectrum of a linear primary at different input intensities. The spectral output is scaled in magnitude with changing input intensity, but the shape and peak location of the spectrum are invariant. This is illustrated by FIG. 3c, which illustrates the lack of effect of changing input intensity on the wavelength of the peak output.

Conversely, FIG. 3b illustrates an output spectrum of a non-linear primary at different input intensities. In addition to the spectral output scaling in magnitude with changing input intensity, the shape and peak location of the spectrum also vary. This is illustrated by FIG. 3d, which illustrates the shifting wavelength of the peak output as a function of input intensity. In the case of the example spectrum of FIG. 3b, the wavelength of the peak output decreases with increasing input intensity. However it will be appreciated that for other non-linear primaries, the input intensity and the wavelength of the peak output may exhibit a different relationship.

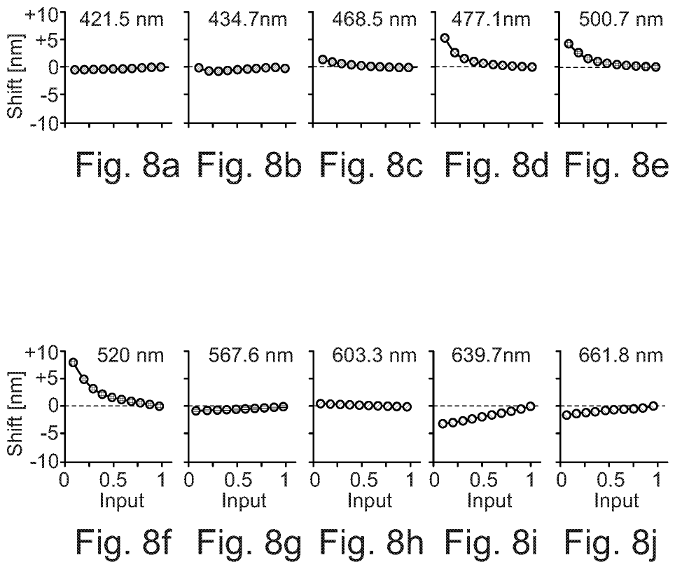

FIG. 8 further illustrates a variation of the spectral shift effect. FIGS. 8a to 8j illustrate the relationship between input intensity and peak spectral location of the output spectrum of eight different example LED primaries with a variety of different peak output wavelengths. As illustrated, the shape of the relationship between intensity and peak location is variable between different primaries and so the precise nature of the spectral shift is unpredictable for each primary unless measured.

Such nonlinearities as illustrated in FIG. 3b and FIG. 8 introduce further complications to relating an output spectrum of a light source 120 given the input intensities of its primaries 121, 122, 123, 124. It has been appreciated that accurately determining a relationship between input intensities and output spectra for a light source 120 can be utilised in accurately determining metameric settings for the light source 120.

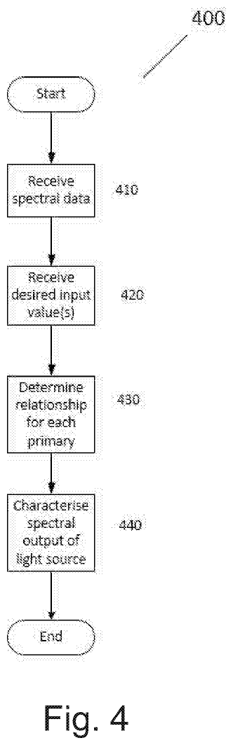

FIG. 4 illustrates a method 400 according to the present invention for determining a relationship between input intensity and spectral output for N primaries 121, 122, 123, . . . , 12N of a non-linear light source 120, without the need to measure the output spectrum for every possible input intensity setting. Aspects of the method 400 may be performed by the processor 112 and the memory devices 111 of the computer 110. Instructions to perform an embodiment of the method 400 may be stored in the one or more memory devices 111.

The method 400 comprises a step 410 of receiving spectral data. The spectral data is indicative of a spectral output of each of the N primaries 121, 122, 123, . . . , 12N at each of a plurality of predetermined intensity values I=[I₁, . . . , Iₙ]. For example, if there are 3 primaries 121, 122, 123 spectral data may be received for a first primary 121 at a plurality of intensity values I₁₂₁=[I_{121,1}, . . . , I_{121,n}], for a second primary 122 at a plurality of intensity values I₁₂₂=[I_{122,1}, . . . , I_{122,n}], and for a third primary 123 at a plurality of intensity values I₁₂₃=[I_{123,1}, . . . , 1_{123,n}]. For each of the N primaries, intensity values I=[I₁, . . . , Iₙ] may be a series of intensity values between predetermined minima and maxima 0 and 1, e.g. I=[0, 0.1, 0.2, 0.3, . . . , 0.9, 1]. Intensity values I may be evenly spaced between 0 and 1, but this is not required. Furthermore, any number of intensity values I may be used. The spectral data may be received at the computer 110 via the one or more networks or may be accessed from memory devices 111.

The spectral data may comprise an output spectrum as a function of wavelength for each of the N primaries 121, 122, 123, . . . , 12N. For example, if there are 3 primaries 121, 122, 123 (e.g. RGB) the received spectral data for each intensity value I₁, . . . , Iₙ may comprise the spectra S₁₂₁(λ), S₁₂₂(λ) and S₁₂₃(λ) taken at each input intensity I₁, . . . , Iₙ for each of the three primaries respectively.

For example, the predetermined intensity values received may be the same 11 equally spaced values for each of the primaries, i.e. I₁₂₁=I₁₂₂=I₁₂₃=[0, 0.1, 0.2, . . . , 1]. Correspondingly, the spectral data received may comprise spectral measurements S₁₂₁(λ; I₁₂₁), S₁₂₂(λ; I₁₂₂) and S₁₂₃(λ; I₁₂₃) taken at each input intensity 0, 0.1, 0.2, . . . , 1.

Method 400 may optionally comprise a step 420 of receiving one or more desired input intensities. The desired input intensities may be one or more input intensities of particular interest, as will be explained. For example, method 400 may be performed to specifically determine the spectral output of the light source 120 at the one or more desired input intensities for each of the primaries 121, 122, 123.

Method 400 comprises a step 430 of determining, in dependence on the received data, a relationship for a primary 121 between intensity and spectral output. The relationship may be determined over a portion of the visible spectrum.

In some embodiments, step 430 comprises determining a further spectral output at one or more intensity values different to the plurality of predetermined intensity values I=[I₁, . . . , Iₙ]. For example, step 430 may comprise determining a further spectral output at the one or more desired input intensities received in step 420. A further spectral output at step 430 may be inferred for each of the N primaries 121, 122, 123, . . . , 12N.

In some embodiments, the further spectral output may be inferred at step 430 by performing an interpolation between at least two of the spectral outputs received in step 410 corresponding to two of the predetermined input intensity values Iₙ. The at least two spectral outputs may be chosen for the interpolation as the desired input intensity value lies between the two predetermined intensity values, i.e. the desired input intensity value lies between two adjacent predetermined intensity values.

A variety of interpolation techniques may be used, for example linear interpolation, polynomial interpolation, or spline interpolation.

For example in step 430, a further spectral output at I=0.15 may be inferred for the primary 121 from the received spectral measurements. For example, S₁₂₁(λ; I₁₂₁=0.15) may be inferred by performing an interpolation between the two spectra S₁₂₁(λ; I₁₂₁=0.1) and S₁₂₁(λ; I₁₂₁=0.2). In other embodiments, S₁₂₁(λ; I₁₂₁=0.15) may be inferred by interpolating between other received spectral measurements, for example between S₁₂₁(λ; I₁₂₁=0) and S₁₂₁(λ; I₁₂₁=0.3). More than two of the received spectral measurements may also be used for the interpolation. The interpolation may be also performed for the other primaries, to infer the spectral outputs S₁₂₂(λ; I₁₂₂=0.15) and S₁₂₃(λ; I₁₂₃=0.15). The same interpolation may be performed for each of the primaries, or different interpolation or inference techniques may be used for each primary.

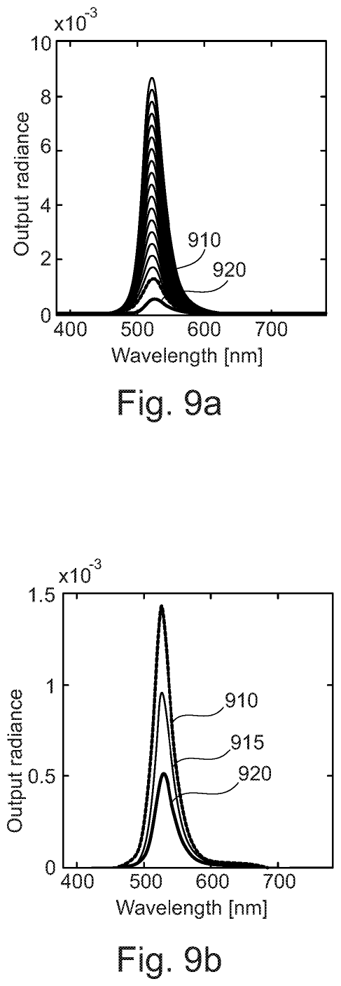

FIG. 9 illustrates graphically, for a primary 121, an example spectral output that may be inferred at step 430. FIG. 9a illustrates received spectral data for the primary 121 at predetermined input intensities, including received spectral data 910 and 920. For example, received spectral data 910 may be indicative of the spectral output of the primary 121 at a predetermined intensity value I=0.2, and received spectral data 920 may be indicative of the spectral output of the primary 121 at a predetermined intensity value I=0.1.

FIG. 9b illustrates a further spectral output 915 that may be inferred at step 430. For example, the further spectral output 915 may correspond to the spectral output of the primary 121 at an intensity value between 0.1 and 0.2, such as I=0.15. Spectral output 915 may be inferred by interpolating between the two spectra 910 and 920, as illustrated.

Method 400 may comprise an optional step 440 of characterising a spectral output of the nonlinear light source 120 in dependence on the relationship determined in step 430. The spectral output of the light source 120 may be characterised as a combination of the spectral output inferred for each of the primaries 121, 122, 123. For example, the spectral output of the nonlinear light source 120 may be characterised as a linear combination of each of the primaries. For example, the spectral output of the light source 120 may be characterised as corresponding to the spectrum S_{LS}(λ; [I₁₂₁, I₁₂₂, I₁₂₃])=S₁₂₁(λ; I₁₂₁)+S₁₂₂(λ; I₁₂₂)+S¹²³(λ; I₁₂₃), wherein S_{LS} is the spectral output of the light source 120, S₁₂₁, S₁₂₂ and S₁₂₃ are the spectral outputs of each primary 121, 122, 123, and I₁₂₁, I₁₂₂, and I₁₂₃ are the input intensities of each primary.

In some embodiments, the spectral output of the nonlinear light source 120 may be characterised in step 440 further in dependence on a background spectrum. The background spectrum may be indicative of a backlight or other non-zero component of background noise in addition to the output of the primaries 121, 122, 123. The background spectrum may be determined from data received over the one or more networks or stored in the memory devices 111. For example, the received data may be pre-measured spectra or an estimate of background spectra. The received data may be indicative of spectral measurements taken when the input intensity of each of the primaries 121, 122, 123 is set to zero. For example, in a three primary system the received data may correspond to a measured output spectrum of the light source S_{LS}(λ; [0,0,0]).

Step 440 may comprise combining the background spectrum with the spectral output inferred for each of the primaries 121, 122, 123 to better characterise the spectral output of the light source 120.

In some embodiments, the spectral output of the nonlinear light source 120 may be characterised in step 440 further in dependence on one or more characteristics of a surface illuminated by the light source 120. The surface one or more characteristics may comprise for example a parameter indicative of a reflectance of a surface. Advantageously, utilising the one or more characteristics of a surface in step 440 allows the method to more accurately characterise the spectral output of the nonlinear light source 120 that reaches a viewer's eye when the nonlinear light source 120 is used to illuminate samples, objects or materials, which will modify the spectral output based on their specific reflectance properties. Step 440 may comprise calibrating the nonlinear light source by measuring reflected light from the surface.

In some embodiments, the spectral output of the nonlinear light source 120 may be characterised in step 440 further by determining a parameter characterising an appearance of an object under illumination of the light source, such as its Colour Rendering Index (CRI) or another similar parameter.

The relationship determined from method 400 between input intensity and spectral output for a light source 120 may be useful in determining metameric settings for the light source 120.

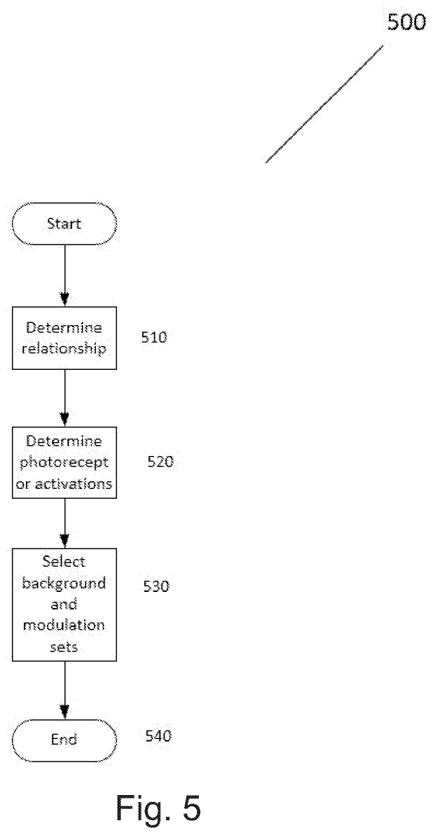

FIG. 5 illustrates a method 500 for determining metameric settings for the light source 120. Aspects of method 500 may be performed by the processor 112 and the one or more memory devices 111 of the computer 110. Instructions to perform an embodiment of the method 500 may be stored in the memory devices 111. Given a light source 120 with N primaries 121, 122, 123, . . . , 12N, method 500 comprises selecting a background and a modulation set of input intensities for each of the N primaries. The background and modulation set of input intensities may be selected to cause the spectral output of the light source 120 to behave in a desired manner, for example by differently activating one or more types of photoreceptors.

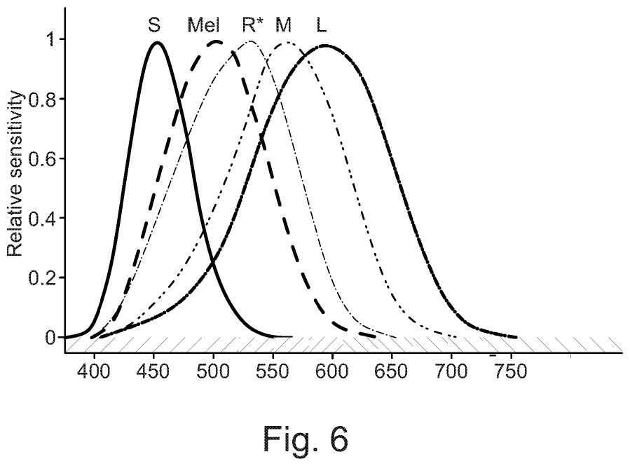

A human retina comprises different types of photoreceptor cells exhibiting different spectral sensitivities. FIG. 6 shows an example illustration of the spectral sensitivity of 5 different types of photoreceptors present in the human retina: S (short wavelength cones), M (medium wavelength cones), L (long wavelength cones), R (rods) and Mel (photoreceptor cells expressing melanopsin). FIG. 6 illustrates the relative sensitivity of each of these photoreceptor types at different wavelengths of light. The sensitivity may be understood as how much the photoreceptor is activated by light as the wavelength of light varies. Each photoreceptor type has a characteristic spectral sensitivity as a function of wavelength. The different photoreceptor types each exhibit a peak sensitivity at a different wavelength of light, although the sensitivities also overlap at some wavelengths.

Method 500 comprises a step 510 of determining a relationship for each of the N primaries between intensity and spectral output over at least a portion of the visible spectrum. Step 510 may comprise, for example, performing method 400.

Method 500 comprises a step 520 of determining a first activation corresponding to activation of a first photoreceptor type and a second activation corresponding to activation of a second photoreceptor type. Step 520 may be performed in dependence on the determined relationship for each of the N primaries, as will be explained.

Method 500 comprises a step 530 of selecting a background and a modulation set of intensity values for each of the N primaries 121, 122, 123, . . . , 12N.

Step 530 may comprise determining a first difference indicative of the difference in activation of the first photoreceptor type between when the light source is set to the background set of intensity values and when the light source is set to the modulation set of intensity values. A first activation corresponding to the background set of intensity values may be determined as the activation of the first photoreceptor type when the N primaries are set to the background set of intensity values. A first activation corresponding to the modulation set of intensity values may be determined as the activation of the first photoreceptor type when the N primaries are set to the modulation set of intensity values. The first difference may then be a difference between the first activation corresponding to the background set of intensity values and the first activation corresponding to the modulation set of intensity values. Step 530 may comprise determining whether the first difference meets one or more criteria, as will be explained.

Step 530 may comprise determining a second difference indicative of the difference in activation of the second photoreceptor type between when the light source is set to the background set of intensity values and when the light source is set to the modulation set of intensity values. A second activation corresponding to the background set of intensity values may be determined as the activation of the second photoreceptor type when the N primaries are set to the background set of intensity values. A second activation corresponding to the modulation set of intensity values may be determined as the activation of the second photoreceptor type when the N primaries are set to the modulation set of intensity values. The second difference may then be a difference between the second activation corresponding to the background set of intensity values and the second activation corresponding to the modulation set of intensity values. Step 530 may comprise determining whether the second difference meets one or more criteria, as will be explained.

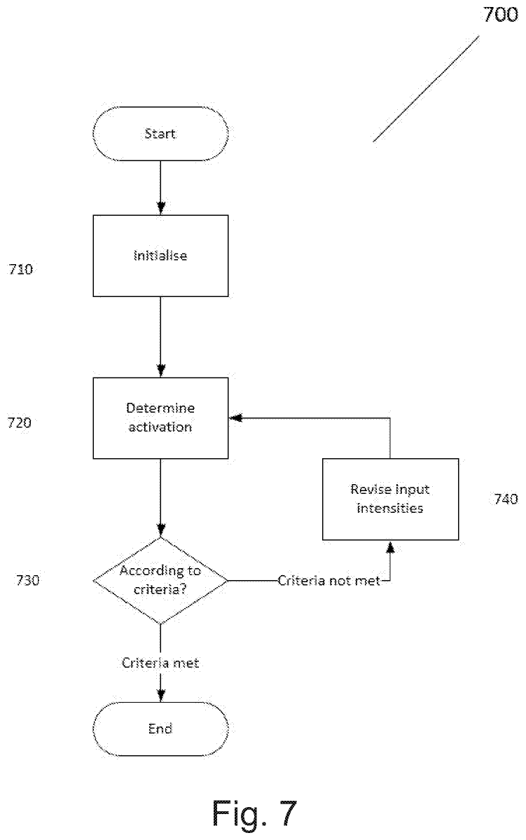

Selecting the background set and the modulation set as in step 530 may comprise selecting initial intensity values for the background set and the modulation set, determining the first difference and the second difference, and revising the background and the modulation set in dependence on the one or more criteria. This implementation of step 530 will be described in more detail with reference to the implementation of method 500 illustrated in FIG. 7.

According to some embodiments of the invention, method 500 may be implemented as illustrated in FIG. 7. FIG. 7 illustrates one embodiment of method 500, referred to as method 700. Aspects of the method 700 may be implemented on computer 110.

Instructions to perform an embodiment of the method 700 may be stored in the memory devices 111.

Method 700 comprises a step 710 of initialising one or more settings. Step 710 comprises initialising a plurality of input intensities, including at least a background and a modulation set of input intensities. An initial background and modulation input intensity may be chosen for each of the N primaries 121, 122, 123, . . . , 12N of the light source 120. For example, step 710 may comprise selecting a set of values for the background input intensities for each of the N primaries I_{BG}=(I_{1,BG}, . . . , I_{N,BG}) and a set of values for the modulation input intensities for each of the N primaries I_{M}=(I_{1,M}, . . . , I_{N,M}). The initial background and modulation input intensities may be selected at random, for example from a random number generator, or may be predetermined.

In some embodiments step 710 may comprise receiving one or more further settings for method 700. For example, step 710 comprises receiving information indicative of the spectral sensitivities of at least two photoreceptor types. The at least two photoreceptor types comprise a first photoreceptor type and a second photoreceptor type. The first photoreceptor type may be a non-visual photoreceptor. For example, the first photoreceptor type may express the photopigment melanopsin. It may be particularly desired to control melanopsin activation, as melanopsin activation is important in the modulation of melatonin production, circadian phase shifting and alertness. The second photoreceptor type may be a visual photoreceptor, for example the second photoreceptor type may comprise one or more cone cells, or rod cells.

It will be appreciated that the first photoreceptor type is not required to be a non-visual photoreceptor. In some embodiments, the first photoreceptor type may be a visual photoreceptor. For example, it may be desired to modulate activation of an S (short wavelength) cone, whilst keeping activation of at least one other photoreceptor constant. The first photoreceptor type may be an S cone. The second photoreceptor type may also be a visual photoreceptor, for example one or more further cone cells or rod cells, or a non-visual photoreceptor.

Step 710 may comprise receiving information indicative of one or more criteria for the activation of the photoreceptors, as will be explained with reference to step 730. Some of the information may be received at the computer 110 over the one or more networks, some of the information may be received through interface 113 for example as user instructions, or some of the information may be accessed from memory devices 111.

Step 710 may comprise receiving one or more desired criteria or parameters for the light source 120. In some embodiments, step 710 may comprise receiving a desired Colour Rendering Index (CRI), or another colour rendition parameter, for the characterised spectral output of the light source 120.

Method 700 comprises a step 720, corresponding to an implementation of step 520, of determining an activation of the first and second photoreceptor types. Step 720 may first comprise determining a relationship for each primary 121, 122, 123 between intensity and spectral output, for example by performing an embodiment of the method 400. The initial background and modulation input intensities may be received at step 420 of the method 400. By performing an embodiment of the method 400, the spectral output of the light source 120 at the initial background and modulation input intensities may then be characterised.

With the characterised spectral output of the light source 120 at the initial background and modulation input intensities, the activation of the first and second photoreceptor types at the initial background and modulation input intensities may be determined at step 720.

Step 720 may comprise determining the activation of each of the first and second photoreceptor types as a weighted sum of the spectral sensitivity of the photoreceptor type and the characterised spectral output of the light source at each of the background and modulation input intensities. For example, the activation P_{1,BG} of the first photoreceptor type at the initial background input intensities may be determined as:

$${P}_{1 , BG} = {\sum }_{λ = 380}^{780} {E}_{1} ( λ ) {S}_{LS} ( λ , {I}_{BG} = ( {I}_{1 , BG} , … , {I}_{N , BG} ) )$$

Wherein E₁(λ) corresponds to the spectral sensitivity of the first photoreceptor type, S_{LS} corresponds to the characterised spectral output of the light source from the method 400, and I_{BG}=(I_{1,BG}, . . . , I_{N,BG}) correspond to the initial background input intensities of each of the N primaries as received in step 710. The weighted sum is shown over wavelengths λ=380 to 780 nm, although a variety of different wavelength ranges may be used for the weighted sum which may result in slight variations in the determined activation. Step 720 may further comprise determining an activation P_{1,M} of the first photoreceptor type at the initial modulation input intensities in the same way, replacing I_{BG}=(I_{1,BG}, . . . , I_{N,BG}) with the initial modulation input intensities I_{M}=(I_{1,M}, . . . , I_{N,M}) received in step 710.

Correspondingly, the activation P₂ of the second photoreceptor type may be determined at each of the initial background and modulation input intensities. For example, at the initial background input intensities P₂ may be determined as:

$${P}_{2 , BG} = {\sum }_{λ = 380}^{780} {E}_{2} ( λ ) {S}_{LS} ( λ , {I}_{BG} = ( {I}_{1 , BG} , … , {I}_{N , BG} ) )$$

The activation P_{2,BG} and P_{2,M} may be determined analogously to the first activation but replacing E₁(λ) with E₂(λ), corresponding to the spectral sensitivity of the second photoreceptor type.

Method 700 comprises a step 730 of determining whether the activations of the first and second photoreceptor types determined in step 720 meet one or more criteria.

Step 730 may comprise determining a first difference between the determined first activation corresponding to the background set and the determined first activation corresponding to the modulation set. The first difference may be determined as a function of P_{1, BG} and P_{1, M}, for example P_{1, M}−P_{1, BG}. Step 730 may further comprise determining a normalised contrast between P_{1, BG} and P_{1, M}, for example:

$${C}_{1} = \frac{{P}_{1 , M} - {P}_{1 , bg}}{{P}_{1 , bg}}$$

Step 730 may comprise determining a second difference between the determined second activation corresponding to the background set and the determined second activation corresponding to the modulation set. The first difference may be determined as a function of P_{2, BG} and P_{2, M}, for example P_{2, M}−P_{2, BG}. Step 730 may further comprise determining a normalised contrast between P_{2, BG} and P_{2, M}, for example:

$${C}_{2} = \frac{{P}_{2 , M} - {P}_{2 , bg}}{{P}_{2 , bg}}$$

Step 730 comprises determining whether the first and second differences meet one or more criteria.

As mentioned, one or more criteria may be received in step 710. The one or more criteria may be chosen in order to select input settings that will result in metamers for the light source 120. For example, it may be desired to achieve a contrast in activation for the first photoreceptor type but achieve consistency in activation for the second photoreceptor type.

The criteria may then comprise a first criteria corresponding to maximising the contrast C₁, if maximal contrast in activation is desired for the first photoreceptor type. In other embodiments, the first criteria may correspond to minimising the difference between the contrast C₁ and a predetermined target contrast Cₛ, if a specific contrast is desired. For example, the first criteria may correspond to minimising the function (Cₛ−C₁)².

The criteria may comprise a second criteria corresponding to minimising the second difference. The second criteria may comprise the second difference being substantially equal to zero, or the second difference being within a pre-defined maximum tolerance limit. In some embodiments, the pre-defined maximum tolerance limit may be determined in dependence on a tolerance parameter. For example, the tolerance parameter may be defined in terms of one or more of photoreceptor contrast, absolute photoreceptor activation, chromaticity tolerances such as variable-step MacAdam ellipses, or luminance tolerances.

The criteria may comprise one or more further criteria or parameters for the light source 120, for example the characterised spectral output of the light source 120 having a desired Colour Rendering Index (CRI) or other colour rendition parameter.

If the criteria are determined not to have been met in step 730, the method may proceed to step 740. Step 740 comprises modifying the background and modulation input intensities and proceeding to return to step 720 implemented with the revised background and modulation input intensities in place of the initial background and modulation input intensities. Whether and how to proceed to step 740 and modify the background and modulation input intensities may be determined by an optimisation algorithm taking as an input the one or more criteria. Any standard constrained optimisation algorithm may be used, for example sequential quadratic programming or interior-point methods.

If the criteria are determined to have been met in step 730, the background and modulation sets of input intensities may then be selected as defined by step 530. The selected background and modulation sets may then be communicated to the light source 120. In other embodiments, the selected background and modulation sets may be stored in memory devices 111, or communicated to a user through interface 113 or over the one or more networks.

It will be appreciated that embodiments of the present invention can be realised in the form of hardware, software or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are embodiments of machine-readable storage that are suitable for storing a program or programs that, when executed, implement embodiments of the present invention. Accordingly, embodiments provide a program comprising code for implementing a system or method as claimed in any preceding claim and a machine readable storage storing such a program. Still further, embodiments of the present invention may be conveyed electronically via any medium such as a communication signal carried over a wired or wireless connection and embodiments suitably encompass the same.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The claims should not be construed to cover merely the foregoing embodiments, but also any embodiments which fall within the scope of the claims.

1. A computer-implemented method of determining metameric settings for a nonlinear light source comprising a set of N primaries, the method comprising:
receiving spectral data indicative of a spectral output of each of the N primaries at each of a plurality of predetermined intensity values;
determining, in dependence on the received data, a relationship for each of the N primaries between intensity and spectral output over at least a portion of a visible spectrum;
determining a first activation corresponding to activation of a first photoreceptor type and a second activation corresponding to activation of a second photoreceptor type in dependence on the determined relationship for each of the N primaries; and
selecting a background set of intensity values and a modulation set of intensity values of the N primaries such that:
a difference between the determined first activation corresponding to the background set and the determined first activation corresponding to the modulation set is according to a first criteria; and
a difference between the determined second activation corresponding to the background set and the determined second activation corresponding to the modulation set is according to a second criteria.

2. The method of claim 1, wherein the first photoreceptor type is a nonvisual photoreceptor.

3. The method of claim 2, wherein the nonvisual photoreceptor expresses the photopigment melanopsin.

4. The method of claim 1, wherein the second photoreceptor type is a visual photoreceptor.

5. The method of claim 1, wherein determining a relationship between intensity and spectral output comprises inferring a further spectral output of each of the N primaries at one or more intensity values further to the plurality of predetermined intensity values.

6. The method of claim 5, wherein inferring the further spectral output comprises performing an interpolation on the spectral output between at least two of the predetermined intensity values.

7. The method of claim 1, further comprising characterising a spectral output of the nonlinear light source in dependence on the determined relationship between intensity and spectral output for each of the N primaries.

8. The method of claim 7, wherein characterising the spectral output of the nonlinear light source is further in dependence on a background spectrum.

9. The method of claim 7, wherein characterising the spectral output of the nonlinear light source is further in dependence on a characteristic of a surface illuminated by the source.

10. The method of claim 7, wherein:
determining the first activation of the first photoreceptor type comprises weighting the spectral output of the nonlinear light source with a spectral sensitivity of the first photoreceptor type; and
determining the second activation of the second photoreceptor type comprises weighting the spectral output of the nonlinear light source with a spectral sensitivity of the second photoreceptor type.

11. The method of claim 1, wherein the second criteria comprises the second difference being substantially equal to zero.

12. The method of claim 1, wherein the second criteria comprises the second difference being within a pre-defined maximum tolerance limit.

13. The method of claim 1, further comprising determining a contrast C between the determined first activation corresponding to the background set and the determined first activation corresponding to the modulation set.

14. The method of claim 13, wherein the contrast C is defined as
$$C = \frac{{P}_{mod} - {P}_{bg}}{{P}_{bg}}$$
wherein P_{mod} is the first activation corresponding to the modulation set and P_{bg} is the first activation corresponding to the background set.

15. The method of claim 13, wherein the first criteria corresponds to maximising the contrast C.

16. The method of claim 13, wherein the first criteria corresponds to minimising the difference between the contrast C and a predetermined target contrast Cₛ.

17. The method of claim 16, wherein the first criteria corresponds to minimising the function (Cₛ−C)², wherein C is the contrast and Cₛ is the predetermined target contrast.

18. The method of claim 1, wherein selecting the background set and the modulation set comprises:
selecting initial intensity values for the background set and the modulation set;
determining the first difference and the second difference; and
revising the background and the modulation set in dependence on the first criteria and the second criteria.

19. (canceled)

20. An apparatus for determining metameric settings for a nonlinear light source comprising a set of N primaries, the apparatus comprising:
an input for receiving spectral data indicative of a spectral output of each of the N primaries at each of a plurality of predetermined intensity values;
one or more processors; and
a memory storing computer executable instructions therein which, when executed by the one or more processors, cause the one or more processors to:
determine, in dependence on the received data, a relationship for each of the N primaries between intensity and spectral output over at least a portion of a visible spectrum;
determine a first activation corresponding to activation of a first photoreceptor type and a second activation corresponding to activation of a second photoreceptor type in dependence on the determined relationship for each of the N primaries; and
select a background set of intensity values and a modulation set of intensity values of the N primaries such that:
a difference between the determined first activation corresponding to the background set and the determined first activation corresponding to the modulation set is according to a first criteria; and
a difference between the determined second activation corresponding to the background set and the determined second activation corresponding to the modulation set is according to a second criteria.

21. A computer-readable data storage medium storing computer-readable instructions which, when executed by one or more processors, perform a method of determining metameric settings for a nonlinear light source comprising a set of N primaries, comprising steps of:
receiving spectral data indicative of a spectral output of each of the N primaries at each of a plurality of predetermined intensity values;
determining, in dependence on the received data, a relationship for each of the N primaries between intensity and spectral output over at least a portion of a visible spectrum;
determining a first activation corresponding to activation of a first photoreceptor type and a second activation corresponding to activation of a second photoreceptor type in dependence on the determined relationship for each of the N primaries; and
selecting a background set of intensity values and a modulation set of intensity values of the N primaries such that:
a difference between the determined first activation corresponding to the background set and the determined first activation corresponding to the modulation set is according to a first criteria; and
a difference between the determined second activation corresponding to the background set and the determined second activation corresponding to the modulation set is according to a second criteria.
